(19) United States
(12) Patent Application Publication
(10) Pub. No.: US 2008/0207748 A1
(43) Pub. Date: Aug. 28, 2008

(54) VITAMIN C PREPARATION

(75) Inventor: Pedro P. Perez, Mount Sinai, NY (US)

Correspondence Address: DARBY & DARBY P.C., P.O. BOX 770, Church Street Station, New York, NY, 10008-0770, US

(73) Assignee: INNOVATION LABS, INC., Mount Sinai, NY (US)

(21) Appl. No.: 12/035,987

(22) Filed: Feb. 22, 2008

Related U.S. Application Data
(60) Provisional application No. 60/902,762, filed on Feb. 22, 2007.
(60) Provisional application No. 60/904,468, filed on Mar. 2, 2007.
(60) Provisional application No. 60/904,593, filed on Mar. 2, 2007.

(51) Int. Cl. A61K 31/375; A61P 25/00

(52) U.S. Cl. 514/474

(57) ABSTRACT

The present invention relates to vitamin C preparations which enhance absorption of vitamin C into cells, and prolong the retention of vitamin C within the blood plasma and tissue of mammals, such as humans. The vitamin C preparations of the present invention include lipophilic molecules which improve the absorption of vitamin C resulting in higher plasma and cellular levels.

The present application claims the benefit of (i) U.S. Provisional Application No. 60/902,762, filed Feb. 22, 2007, (ii) U.S. Provisional Application No. 60/904,468, filed Mar. 2, 2007, and (iii) U.S. Provisional Application No. 60/904,593, filed Mar. 2, 2007, all of which are hereby incorporated by reference.

### FIELD OF THE INVENTION

The present invention relates to vitamin C preparations having enhanced bioavailability.

### BACKGROUND OF THE INVENTION

According to the National Institute of Health and the Food and Nutritional Board of the National Academy of Science, vitamin C is an essential nutrient involved in many biological functions. Vitamin C can only be acquired through diet (i.e., food or nutritional supplement).

Vitamin C has been implicated as an important dietary component as it is required for physiological and metabolic activities including the development of healthy neurons (Zhou et al., 2003; Weeks & Perez, 2007), prevention of neurodegenerative diseases (Boothby & Doering, 2005; Landmark 2006), wound healing (Kaplan et al., 2004; Marionnet C et al., 2006; Weeks & Perez, 2007), and the maintenance of a healthy immune system (Fay et al., 1994; Lehr et al., 1994; Weeks & Perez, 2007).

Given the importance of vitamin C, the bioavailability of vitamin C has been the focus of intense research. An improvement in absorption and retention of vitamin C in blood plasma or tissue would increase the beneficial effects of vitamin C. Thus, there is a continuing need for vitamin C preparations having enhanced bioavailability.

### SUMMARY OF THE INVENTION

The present invention relates to vitamin C preparations which enhance absorption of vitamin C into cells, and prolong the retention of vitamin C within the blood plasma and tissue of mammals, such as humans. The vitamin C preparations of the present invention include lipophilic molecules which improve the absorption of vitamin C resulting in higher plasma and cellular levels.

One embodiment of the invention is a vitamin C preparation comprising:
(a) at least about 90% by weight of vitamin C,
(b) at least about 0.1% by weight of lipophilic molecules comprising (i) one or more saturated straight C₃₀-C₃₄ fatty alcohols, (ii) one or more unsaturated ω-9 C₁₈-C₂₄ fatty acids, (iii) optionally one or more saturated straight C₁₄-C₂₀ fatty acids, (iv) optionally one or more unsaturated ω-3 C₁₆-C₂₄ fatty acids, and (v) optionally one or more unsaturated ω-6 C₁₈-C₂₂ fatty acids; and
(c) optionally at least about 0.1% by weight of bioflavonoids,
   based upon 100% total weight of the vitamin C preparation. The preparation preferably comprises at least 0.1% by weight of component (b)(i) (e.g., about 0.5 to about 4% by weight of component (b)(i)), and at least 0.01% by weight of component (b)(ii), and when the preparation includes one or more of components (b)(iii)-(b)(v), each included component is present in an amount of at least 0.01% by weight. The vitamin C preparation is preferably in the form of an oral dosage form, such as a tablet or capsule.

Preferably, the amount of the lipophilic molecules in the vitamin C preparation ranges from 0.1 or 0.2 to 5% by weight, based upon the total weight of the preparation. According to one preferred embodiment, the vitamin C preparation contains about 0.8 to about 1.8% by weight of the lipophilic molecules and even more desirably about 1 to about 1.5% by weight of the lipophilic molecules.

The vitamin C preparation can be administered to a person to (i) promote a healthy nervous system, (ii) prevent or decrease the risk of developing a neurodegenerative disease, (iii) enhance NGF-mediated neurite outgrowth, (iv) promote wound healing, (v) enhance fibroblast adhesion to and the interaction with the extracellular matrix, (vi) protect the immune system from xenobiotics, (vii) decrease the risk of developing an oxidative pathogenesis, and (viii) decrease the risk of developing cancer, cardiovascular diseases, atherosclerosis, and other age-related diseases associated with cytotoxic, genotoxic, and proinflammatory mechanisms. According to one embodiment, the method includes:

(a) recognizing the vitamin C preparation as being effective for one of the aforementioned purposes (e.g., to promote a healthy nervous system) and optionally that the person is in need thereof, and

(b) after such recognition, orally administering to the human an effective amount of the vitamin C preparation.

### BRIEF DESCRIPTION OF THE DRAWINGS

FIG. 1 is a graph of the concentration of vitamin C in H9 human T-cells as measured 15-120 minutes following administration of the vitamin C preparation of Example 1 (PWC), ascorbic acid (AA), calcium ascorbate (CaA), or calcium ascorbate-calcium threonate-dehydroascorbate (Ester-C) (commercially available as Ester-C® from Nature's Value of Coram, N.Y.) (Ester-C®).

FIG. 2 is a graph of the percentage inhibition of 1,1-diphenyl-2-picryl hydrazyl (DPPH) reduction as measured by the procedure described in Example 4 following administration of 1, 2.5, 5, 10, or 20 μg/ml of the vitamin C preparation of Example 1.

FIG. 3 is a graph of the percentage of cells exhibiting neurite outgrowth over 24 hours following administration of vehicle (-) or 0.5 μM of the vitamin C preparation of Example 1 (PWC), ascorbic acid (AA), calcium ascorbate (CaA), or calcium ascorbate-calcium threonate-dehydroascorbate (EsterC) or a control, as measured by the procedure described in Example 5.

FIG. 4 is a graph showing the percentage of fibroblasts adhered to fibronectin substrates following administration of vehicle (-) or 50 μM of the vitamin C preparation of Example 1 (PWC), ascorbic acid (AA), calcium ascorbate (CaA), or calcium ascorbate-calcium threonate-dehydroascorbate (EsterC) or a control as measured by the procedure described in Example 6.

### DETAILED DESCRIPTION OF THE INVENTION

### Definitions

The term “vitamin C,” unless otherwise stated, refers to ascorbic acid and pharmaceutically acceptable salts thereof, including, but not limited to, mineral salts of ascorbic acid, effervescent vitamin C (e.g., a combination of ascorbic acid, citric acid and sodium bicarbonate), chelates of ascorbic acid, and alkaline salts of ascorbic acid.

### Lipophilic Molecules

Suitable lipophilic molecules include, but are not limited to, those derived from natural waxes such as, but is not limited to, sugar cane wax, rice bran wax, carnauba wax, candelilla wax, japan wax, ouricury wax, bayberry wax, shellac wax, sunflower wax, orange wax, and beeswax. According to one preferred embodiment, the lipophilic molecules are derived from rice bran wax, carauba wax, cadelilla wax, and beeswax. According to a more preferred embodiment, the lipophilic molecules are derived from rice bran wax. Suitable lipophilic molecules extracted from natural waxes include, but are not limited to, palmitic acid, linoleic acid, linolenic acid, oleic acid, and steric acid.

According to one preferred embodiment, the vitamin C preparation includes one or more or all of the following lipophilic molecules at the recited weight ratios:

about 0.1-3.0 units (by weight) palmitic acid,

about 0.1-20.0 units linoleic acid,

about 0.1-6.0 units alpha linolenic acid,

about 0.1-4.0 units oleic acid,

about 0.1-8.0 units steric acid,

about 0.1-0.9 units arachidic acid,

about 0.1-0.9 units heneicosanoic acid,

about 1.0-9.0 units behenic acid,

about 1.0-9.0 units tricosanoic acid,

about 0.1-9.0 units lignoceric acid,

about 0.5-9.0 units cerotic acid,

about 1.0-10.0 units heptacosanoic acid,

about 0.5-15.0 units montanic acid,

about 2.0-26.0 units melissic acid,

about 0.5-16.0 units docosahexaenoic acid,

about 0.5-9.0 units docosapentaenoic acid,

about 0.5-19.0 units docosatetraenoic acid,

about 0.5-9.0 units docosadienoic acid,

about 0.1-18.0 units erucic acid,

about 0.1-0.9 units nervonic acid,

about 0.1-80.0 units cetyl alcohol-hexadecanol-palmityl alcohol,

about 0.1-50.0 units 1-heptadecanol,

about 0.1-10.0 units 1-eicosanol-arachidyl alcohol,

about 0.1-30.0 units 1-docosanol-behenyl alcohol,

about 10.0-150.0 units lignoceryl alcohol-1-tetracosanol,

about 10.0-120.0 units 1-hexacosanol-ceryl alcohol,

about 0.1-20.0 units 1-heptacosanol,

about 5.0-200.0 units 1-octacosanol,

about 150.0-400.0 units 1-triacontanol-melissyl alcohol,

about 100.0-200.0 units dotriacontanol, and

about 50.0-150.0 units tetratriacontanol.

In one embodiment, the vitamin C preparation includes one or more or all of the following lipophilic molecules at the recited weight percentages:

about 0.01-0.3% (by weight) palmitic acid,

about 0.01-2.0% linoleic acid,

about 0.01-0.6% alpha linolenic acid,

about 0.01-0.4% oleic acid,

about 0.1-0.8% steric acid,

about 0.01-0.09% arachidic acid,

about 0.01-0.09% heneicosanoic acid,

about 0.1-0.9% behenic acid,

about 0.1-0.9% tricosanoic acid,

about 0.01-0.9% lignoceric acid,

about 0.05-0.9% cerotic acid,

about 0.1-1.0% heptacosanoic acid,

about 0.05-1.5% montanic acid,

about 0.2-2.6% melissic acid,

about 0.05-1.6% docosahexaenoic acid,

about 0.05-0.9% docosapentaenoic acid,

about 0.05-1.9% docosatetraenoic acid,

about 0.05-0.9% docosadienoic acid,

about 0.01-1.8% erucic acid,

about 0.01-0.09% nervonic acid,

about 0.01-8.0% cetyl alcohol-hexadecanol-palmityl alcohol,

about 0.01-5.0% 1-heptadecanol,

about 0.01-1.0% 1-eicosanol-arachidyl alcohol,

about 0.01-3.0% 1-docosanol-behenyl alcohol,

about 1.0-15.0% lignoceryl alcohol-1-tetracosanol,

about 1.0-12.0% 1-hexacosanol-ceryl alcohol,

about 0.01-2.0% 1-heptacosanol,

about 0.5-20.0% 1-octacosanol,

about 15.0-40.0% 1-triacontanol-melissyl alcohol,

about 10.0-20.0% dotriacontanol, and

about 5.0-15.0% tetratriacontanol, based upon 100% total weight of the lipophilic molecules in the vitamin C preparation.

The mixture of lipophilic molecules can be obtained by (1) saponification of a wax (e.g., a natural wax), (2) solidifying and grinding the saponified wax to a d₉₀ less than 2000 microns (e.g., 100-500 microns or 500-2000 microns), (3) extracting the ground material with acetone or an alcohol (e.g., ethanol or isopropanol), and (4) optionally solidifying and grinding the extracted material to a d₉₀ less than 2000 microns (e.g., 100-500 microns or 500-2000 microns).

The natural waxes undergo saponification or hydrolysis before the extraction procedure. For saponification, the natural waxes are heated using a jacketed kettle at 90° C. for 3 hours until the wax was completely melted, KOH is added, and the mixture is held at 90° C. for 1 hour with stirring. For hydrolysis, the natural waxes are heated using a jacketed kettle at 90° C. for 3 hours until the wax was completely melted, sulfuric acid aqueous solution is added, and mixture is held at 90° C. for 1 hour with stirring. After 1 hour of stirring the saponified or hydrolyzed wax is poured into cart trays and dried at 21.1° C. before undergoing the extraction procedure.

The extraction of the natural waxes may be performed by either dispersed-solids extraction or immersion type percolation extraction. For the dispersed-solids extraction, the natural waxes are ground to a particle mesh size of 100 to 425 microns and subjected to liquid extraction in a dispersed-solids extraction system. In the case of immersion type percolation extraction, the natural waxes are ground to a particle mesh size of 500 to 2000 microns and subjected to liquid extraction in a solid-liquid immersion type percolating extractor system. In both types of extraction equipment, the natural mixture of aliphatic alcohols, saturated fatty acids, and omega-3, omega-6, omega-9 fatty acids is selectively extracted with adequate hot organic solvents such as acetone and ethanol with a temperature range of 55° C. to 75° C. The extractions are purified with hot organic solvents such as hexane, heptane, and acetone; recovered; and dried. The extractions contain a mixture of aliphatic alcohols having 17 to 34 carbon atoms, saturated fatty acids having 16 to 30 saturated carbon atoms, and omega-3, omega-6, omega-9 fatty acids with melting point between 70 and 80° C. The ratio of the natural wax particles to hot liquid extractants is from 1 to 4 and 1 to 10. According to one preferred embodiment, the extractions with hot organic solvents at 60° C., and the ratio of natural wax particles to hot liquid extractants is 1 to 8.

### Bioflavonoids

Suitable bioflavonoids include, but are not limited to, rutin, naringin, hesperidin, neohesperidin, neohesperidin dihydrochalcone, naringenin, hersperitin, nomilin, and gallic acid. According to one preferred embodiment, the vitamin C preparation contains hesperidin, gallic acid, and optionally other bioflavonoids.

According to one preferred embodiment, the vitamin C preparation includes one or more or all of the following bioflavonoids at the recited weight ratios:

about 20-120 units (by weight) rutin,

about 25-100 units naringin,

about 7000-20000 units hesperidin,

about 5-100 units neohesperidin,

about 10-100 units neohesperidin dihydrochalcone,

about 5-100 units naringenin,

about 5-100 units hersperitin,

about 50-150 units nomilin, and

about 120,000-1,000,000 units gallic acid.

In one embodiment, the vitamin C preparation includes one or more or all of the following bioflavonoids at the recited weight percentages:

about 20-120 ppm rutin,

about 25-100 ppm naringin,

about 7000-20000 ppm hesperidin,

about 5-100 ppm neohesperidin,

about 10-100 ppm neohesperidin dihydrochalcone,

about 5-100 ppm naringenin,

about 5-100 ppm hersperitin,

about 50-150 ppm nomilin, and

about 120-1000 mg/g gallic acid,

based on 1 g of the bioflavonoid mixture.

According to one embodiment, the vitamin C preparation includes vitamin C and the lipophilic molecules at a weight ratio ranging from about 1000:1 to about 10:1. According to a preferred embodiment, the weight ratio ranges from about 100:1 to about 8:1.

According to a preferred embodiment, the vitamin C preparation includes at least about 90% by weight of vitamin C and about 0.1% by weight of the lipophilic molecules based upon 100% total weight of the vitamin C preparation. More preferably, the vitamin C preparation includes from about 90 to about 99% by weight of vitamin C and from about 1 to about 8% by weight of lipophilic molecules. According to another embodiment, the vitamin C preparation includes from about 90 to about 98% by weight of vitamin C and from about 2 to about 7% (e.g., about 5%) by weight of lipophilic molecules.

According to a preferred embodiment, the vitamin C preparation includes at least about 90% by weight of vitamin C, from about 0.1 to about 9% by weight of lipophilic molecules, and from about 0.1 to about 5% by weight of bioflavonoids.

According to another embodiment, the vitamin C preparation includes about 200 to 40,000 IU vitamin C.

### Dosage Forms

The vitamin C preparation is preferably in the form of an oral dosage form, such as beads, pellets, granules, capsules (soft or hard), sachets, tablets, powders, dispersible powders capable of effervescing upon addition of water, aqueous or oily suspensions, emulsions, syrups, elixirs, or lozenges. For example, the oral dosage form can be an chewable tablet or gum, oral liquid dosage form, such as a suspension in an aqueous or non-aqueous liquid solution, or an emulsion which can be a soft drink, tea, milk, coffee, juice, sports drink, or water. The vitamin C preparation can also be incorporated into various products, such as nutritional supplements (including vitamins and multi-vitamins), foods (including health food products such as nutrition bars), and drinks (including fruit juices such as energy drinks).

Generally, the daily dosage of the vitamin C preparation on a vitamin C weight basis can range from 30 mg to 2 g. For instance, the daily dosage can be 60 mg to 1 g or 60 mg to 500 mg. Desirably, the daily dosage ranges from 60 mg to 500 mg (e.g., the daily dosage can be 400 mg). According to one preferred embodiment, the daily dosage ranges from 60 mg to 200 mg (e.g., the daily dosage can be 60, 100, or 200 mg). The daily dose can be achieved by administration of a single dosage form of the invention or alternatively, two or more such dosage forms. Preferably, the daily dose is achieved by administration of only one or two dosage forms (e.g., once daily dosing or b.i.d.). Therefore, the present invention includes, but is not limited to, dosage forms containing 30, 60, 100, 200, 400, 500, or 1000 mg of the vitamin C preparation (on a vitamin C weight basis).

The vitamin C preparation may include one or more excipients or additives. Suitable excipients and additives include, but are not limited to, additional antioxidants (e.g., phenolic compounds), inert diluents (such as lactose, sodium carbonate, calcium phosphate, and calcium carbonates), granulating and disintegrating agents (such as corn starch and algenic acid), binders (such as starch), lubricants (such as magnesium stearate, stearic acid and talc), preservatives (such as ethyl or propyl p-hydroxybenzoate), colorants, flavoring agents, release modifying agents, thickeners, and any combination of any of the foregoing. Suitable antioxidants include, but are not limited to, bioflavonoids, flavonoids, flavonols, flavanones, flavones, flavonals, flavanolols, and flavanols.

Suitable inert solid diluents include, but are not limited to, calcium carbonate, calcium phosphate and kaolin. Suitable diluents for soft capsules include, but are not limited to, water and oils such as peanut oil, liquid paraffin, corn oil, wheat germ oil, soybean oil, and olive oil.

Aqueous suspensions or dispersions contain the vitamin C preparation, for example, in fine powder form together with one or more suspension or dispersion (or wetting) agents. Suitable suspension agents include, but are not limited to, sodium carboxymethylcellulose, methylcellulose, hydroxypropylmethylcellulose, sodium alginate, polyvinyl-pyrrolidone, gum tragacanth and gum acacia. Suitable dispersing or wetting agents include, but are not limited to, lecithin, condensation products of an alkylene oxide with fatty acids, condensation products of ethylene oxide with long chain aliphatic alcohols, condensation products of ethylene oxide with partial esters derived from fatty acids and a hexitol such as polyoxyethylene sorbitol monooleate, or condensation products of ethylene oxide with partial esters derived from fatty acids and hexitol anhydrides.

Dispersible powders and granules suitable for preparation of an aqueous suspension by the addition of water contain the vitamin C preparation, for example, together with a dispersing agent, wetting agent, or suspending agent. Suitable dispersing agents, wetting agents, and suspending agents include those mentioned above.

Oily suspensions may be formulated by suspending the vitamin C preparation in an oil, such as an vegetable oil or a mineral oil. The oily suspensions may also contain a thickening agent such as carnauba wax, candelilla wax, rice bran wax, beeswax, hard paraffin, or cetyl alcohol.

The vitamin C preparation may be in the form of an oil-in-water emulsion. The oily phase may be a vegetable based oil or a mineral based oil. Suitable emulsifying agents include, for example, naturally occurring gums such as acacia and tragacanth gum, naturally occurring phosphatides such as soy bean, lecithin, esters and partial esters derived from fatty acids and hexitol anhydrides and condensation products of partial esters with ethylene oxide, such as polyoxyethylene sorbitan monooleate.

Syrups and elixirs may be formulated with sweetening agents such as glycerol, propylene glycol, sorbitol, aspartame, or sucrose, and may also contain a demulcent, preservative, flavoring, or coloring agent.

The vitamin C preparation may be also in a form suitable for administration by inhalation (e.g., as a finely divided powder or a liquid aerosol), or for parenteral administration (e.g., as a sterile aqueous or oily solution for intravenous, subcutaneous, intramuscular dosing or as a suppository for rectal dosing). Administration of the vitamin C preparation by these non-oral routes avoids gastrointestinal side effects, which may accompany high doses of vitamin C released in the stomach.

The vitamin C preparation can also be delivered topically, for example, to protect the skin from free radicals, promote wound healing (for instance, for healing cuts, abrasions, sun damage (e.g., sun burn), wrinkles, and scars), and/or reduce inflammation. The vitamin C preparation of the invention provides superior penetration of vitamin C through the skin than vitamin C alone. Transdermal delivery of the vitamin C preparation permits systemic delivery of the vitamin C while avoiding gastrointestinal side effects. The topical formulation containing the vitamin C preparation can be in the form of a solution, suspension, lotion, emulsion, ointment, cream, or gel. According to a preferred embodiment, the topical formulation is a cream or lotion. The formulation may include additional active ingredients. These formulations may prepared by methods known in the art, and typically include a topically acceptable vehicle. One embodiment is a topical formulation containing about 0.5 to about 25% by weight of the vitamin C preparation of the present invention, based upon 100% total weight of the topical formulation. For instance, the topical formulation can contain 0.5-2%, 1-2%, 1-5%, 1-10%, 5-15%, 5-20%, or 10-20% by weight of the vitamin C preparation.

The vitamin C preparation could be used to coat a medical device that is then positioned to a desired target location within the body, whereupon the vitamin C preparation elutes from the medical device. Preferably, the coating includes a therapeutically effective amount of the vitamin C preparation. In one embodiment, the medical device is positioned so that the vitamin C preparation is released in a therapeutically effective amount to a targeted site such as a diseased or injured tissue or organ. The device can be introduced temporarily or permanently into a mammal (e.g., a human) for the prophylaxis or therapy of a medical condition, or to augment the immune system. The device can be introduced subcutaneously, percutaneously, or surgically. The medical device can be selected from stents, synthetic grafts, artificial heart valves, artificial hearts and fixtures to connect the prosthetic organ to the vasculature, venous valves, abdominal aortic aneurysm grafts, inferior venal caval filters, catheters including permanent drug infusing catheters, embolic coils, embolic materials used in vascular embolization mesh repair materials, a Dracon vascular particle orthopedic metallic plates, rods, screws, and vascular sutures.

The vitamin C preparation may be formulated to provide immediate release or controlled release (e.g., sustained release) of the vitamin C preparation, for example, to provide effective doses of vitamin C over extended periods of time to prolong the biological activity and beneficial biochemical functions of vitamin C. One embodiment of the invention is a controlled release dosage form (such as a solid dosage form) containing about 200 to 40,000 IU vitamin C, about 1 to 100 mg of lipophilic molecules, and 1 to 500 mg of bioflavanoids. For example, the controlled release dosage form may release about 10 to about 35% by weight of the total vitamin C preparation within about 2 hours in an in vitro dissolution test, and about 40 to about 70% by weight of the total vitamin C preparation within about 8 hours. According to another embodiment, the controlled release dosage form may release about 50% by weight of the total vitamin C preparation within about 2 hours in an in vitro dissolution test, and more than 90% by weight of the total vitamin C preparation within about 6 or 8 hours. Any type of controlled release system known in the art can be used. The in vitro dissolution test is conducted using the Basket Method (Apparatus 1) with 900 ml 0.1N HCl as the medium run at 100 RPM at a temperature of 37° C. The samples are filtered through Whatman filter paper #1 and the amount of vitamin C is calculated based on the equivalence to standard dicholorophenol-indophenol solutions.

Solid controlled release dosage forms (e.g., tablets) can be formulated (e.g., coated) so as to prolong the release of the vitamin C preparation into the gastrointestinal tract, or to prevent the release of the vitamin C preparation in the stomach in order to prevent or attenuate the gastrointestinal side effects which can accompany high doses of vitamin C released in the stomach. For example, the vitamin C preparation can be enteric coated so as to prevent significant release of the preparation in the stomach. Controlled release of the vitamin C preparation can prolong therapeutic and/or immunoprotective systemic concentrations of vitamin C in a person.

One embodiment of the invention is a three layer controlled release dosage form (e.g., a tablet) where each layer contains a vitamin C preparation of the invention. The vitamin C preparation of each layer can be the same or different. At least one of the layers provides controlled release of the vitamin C preparation. For example, the dosage form can include (i) a first layer, (ii) a second layer, and (ii) an outer layer surrounding the first and second layers, where the first layer and outer layer provide controlled release of the vitamin C preparation(s) and the second layer provides immediate release of the vitamin C preparation.

According to one preferred embodiment, the outer layer releases substantially all (>90%) of the vitamin C preparation in a controlled manner within 60, desirably 30, and even more desirably 20 minutes, as determined by the aforementioned in vitro dissolution test. The second layer provides immediate release of the vitamin C preparation contained therein. Finally, the first layer releases the vitamin C preparation contained therein in a controlled manner over at least 6 hours (e.g., substantially of the vitamin C preparation may be released within 6-10 hours or 6-8 hours), as determined by the aforementioned in vitro dissolution test.

Transdermal patch devices can also provide controlled administration (e.g., continuous or other sustained administration) of the vitamin C preparation. Methods for preparing controlled release transdermal formulations are known in the art. For example, the transdermal device may contain an impermeable backing layer which defines the outer surface of the device and a permeable skin attaching membrane, such as an adhesive layer, sealed to the outer layer in such a way as to create a reservoir between them wherein the therapeutic agent is placed (e.g., a bandage or patch (including a time released patch)).

Other suitable controlled release systems include, but are not limited to, long-term sustained implants, aqueous or oily suspensions, emulsions, syrups, elixirs, or lozenges, chewable tablet or gum, foods, beverages, osmotic systems, and dissolution system (e.g., effervescent oral dosage form).

The vitamin C preparation of the present invention is preferably administered orally to a mammal (e.g., a human), but it can also be administered by other routes of administration, such as intravenously or subcutaneously.

### Preparation of Formulation

The vitamin C preparation of the present invention may be prepared by methods well known in the art, such as mixing the vitamin C, lipophilic molecules, optionally bioflavanoids, and any desired excipients.

The following examples illustrate the invention without limitation.

### EXAMPLE 1

### Lipid Metabolite Extraction:

Saponification: 25 kg of rice bran wax was heated using a jacketed kettle at 90° C. for 3 hours until the wax was completely melted. 4.67 L of 8.0 M KOH (450 g/l) in water was slowly added with continuous stirring and heating. The mixture was held at 90° C. for 1 hour with stirring. After 1 hour the saponified wax was poured into cart trays and dried at 21.1° C. The 32.1 kg of cooled dried saponified wax was then ground to a powder (100-425 or 500-2000 microns).

Extraction: 9.6 kg of the saponified wax was placed in 8 extraction thimbles (1.2 kg of saponified wax per extraction thimble). 100 L of acetone were pumped into a 200 L cylindrical-bottom flask and connected to a soxhlet system. The system was refluxed for approximately 24 hours, and the extract was pumped to a jacketed reactor. The extract was chilled to approximately 10° C. with 20 rpm agitation (20 rpm) for 10 hours. The chilled extract was then centrifuged in a vertical basket centrifuge. The collected solid was poured into trays and vacuum dried for 16 hours. The dried solid was then ground to a powder.

### Preparation of the Vitamin C Preparation:

A jacketed mixer was charged with dry powder of 58 kg of vitamin C, 0.75 kg of the lipid metabolites prepared above and 1.5 kg of bioflavonoids (AnMar International Ltd; Bridgeport, Conn.). The mixer was then turned on (agitation is initiated—plows) to create a homogenous mixture of dry powder. The high speed shearing devices (choppers) were initiated for 1 minute. Hot water was then pumped through the jacket of the mixer to heat the mixture to 80° C. with continuous mixing (plows only) for 15 minutes for complete encapsulation. The encapsulated mixture was cooled by running chilled water (10° C.) through the jacket under continuous mixing for 1 hour until a free-flowing powder was formed. The powder was discharged into a double polyethylene-lined container and then passed through a comminuting mill running at approximately 2500 rpm equipped with a 0.15 mm screen. The milled powder was collected into appropriately labeled, double polyethylene-lined drums and reconciled.

TABLE 1
The formulation of a of the invention is shown below:

| Ingredients | Amount | |
|---|---|---|
| Vitamin C | 90-99% | |
| Lipophilic Molecules | 0.1-5% | |
| palmitic acid | 0.1-3.0 | mg/g* |
| linoleic acid (ω-6 fatty acid) | 0.1-20.0 | mg/g |
| alpha linolenic acid (ω-3 fatty acid) | 0.1-6.0 | mg/g |
| oleic acid (ω-9 fatty acid) | 0.1-4.0 | mg/g |
| stearic acid | 0.1-8.0 | mg/g |
| arachidic acid | 0.1-0.9 | mg/g |
| heneicosanoic acid | 0.1-0.9 | mg/g |
| behenic acid | 1.0-9.0 | mg/g |
| tricosanoic acid | 1.0-9.0 | mg/g |
| lignoceric acid | 0.1-9.0 | mg/g |
| cerotic acid | 0.5-9.0 | mg/g |
| heptacosanoic acid | 1.0-10.0 | mg/g |
| montanic acid | 0.5-15.0 | mg/g |
| melissic acid | 2.0-26.0 | mg/g |
| Docosahexaenoic acid (DHA) (ω-3 fatty acid) | 0.5-16.0 | mg/g |
| docosapentaenoic acid (DPA) (ω-3 fatty acid) | 0.5-9.0 | mg/g |
| Docosatetraenoic acid (DTA) (ω-6 fatty acid) | 0.5-19.0 | mg/g |
| docosadienoic acid (ω-6 fatty acid) | 0.5-9.0 | mg/g |
| erucic acid (ω-9 fatty acid) | 0.1-18.0 | mg/g |
| nervonic acid (ω-9 fatty acid) | 0.1-0.9 | mg/g |
| cetyl alcohol-hexadecanol-palmityl alcohol | 0.1-80.0 | mg/g |
| 1-heptadecanol | 0.1-50.0 | mg/g |
| 1-eicosanol-arachidyl alcohol | 0.1-10.0 | mg/g |
| 1-docosanol-behenyl alcohol | 0.1-30.0 | mg/g |
| lignoceryl alcohol-1-tetracosanol | 10.0-150.0 | mg/g |
| 1-hexacosanol-ceryl alcohol | 10.0-120.0 | mg/g |
| 1-heptacosanol | 0.1-20.0 | mg/g |
| 1-octacosanol | 5.0-200.0 | mg/g |
| 1-triacontanol-melissyl alcohol | 150.0-400.0 | mg/g |
| Dotriacontanol | 100.0-200.0 | mg/g |
| Tetratriacontanol | 50.0-150.0 | mg/g |
| Bioflavonoids (optional) | 0.1-5% | |
| Rutin | 20-120 | ppm** |
| Naringin | 25-100 | ppm |
| Hesperidin | 7000-20000 | ppm |
| Neohesperidin | 5-100 | ppm |
| neohesperidin dihydrochalcone | 10-100 | ppm |
| Naringenin | 5-100 | ppm |
| Hersperitin | 5-100 | ppm |
| Nomilin | 50-150 | ppm |

| | |
|---|---|
| gallic acid | At least 120 mg/g |
| | (q.s.) |

*mg/g = mg of component per g of total lipophilic molecules
**ppm or mg/g = ppm or mg of component per g of total bioflavonoid mixture

### EXAMPLE 2

The rate of vitamin C absorption in H9 cells, a human T-cell line, was determined for the formulation of Example 1 and other vitamin C formulations.

Cells from the human T-lymphoblastic H9 cell line were starved of vitamin C for 18 hours in serum-free media and subsequently suspended in 50 μM of (1) ascorbic acid (AA), (2) calcium ascorbate (CaA), (3) calcium ascorbate-calcium threonate-dehydroascorbate (commercially available as Ester-C® from Nature's Value of Coram, N.Y.) (Ester-C®), or (4) the vitamin C preparation of Example 1 (PWC). At the times indicated in FIG. 1, cells were harvested and measured for vitamin C and protein content. The cellular vitamin C levels of the cells were measured using the 2,4-dinitrophenylhydrazine spectrophotometric technique (Bessey et al., 1947).

Over a two hour period, the level of vitamin C uptake from Example 1 was consistently higher than that observed with ascorbic acid, calcium ascorbate, and calcium ascorbate-calcium threonate-dehydroascorbate (See FIG. 1). At fifteen minutes, cellular vitamin C levels ranged from 7±1.4 nmol/mg cellular protein with ascorbic acid, to over double that amount (15±2.4 nmol/mg protein) with the vitamin C preparation of Example 1. The absorbed vitamin C levels rose significantly with time, peaking at approximately two hours with cellular levels ranging from 31 nmol/mg protein for ascorbic acid and 50 nmol/mg protein for the vitamin C preparation of Example 1.

In order for vitamin C to exert its beneficial effects, it must be taken up into the cell. To date, vitamin-C lipid metabolites exhibits the greatest amount of vitamin C uptake and retention as compared to all other vitamin C formulations.

### EXAMPLE 3

The ability to inhibit pesticide-induced T-lymphocyte aggregation was determined for the formulation of Example 1 and other vitamin C formulations.

The human T-lymphoblastic H9 cell line was incubated with vehicle (-) or with one of two activators of T-lymphocyte aggregation, phytohemagglutinin (PHA; 10 μm) or bifenthrin (10 mM). The cells were immediately treated with 0.5 μM of (1) ascorbic acid (AA), (2) calcium ascorbate (CaA), (3) calcium ascorbate-calcium threonate-dehydroascorbate (Ester-C®), or (4) the vitamin C preparation of Example 1 (PWC) for 30 minutes at 37° C. After treatment, the ability of each formulation to inhibit homotypic aggregation was measured by counting aggregate size at 400× magnification.

The vitamin C preparation of Example 1, inhibited the aggregation of the T-lymphocytes induced by the pesticide PHA or the pesticide bifenthrin by 88% and 84% respectively (Table 2). The reduction in T-lymphocyte aggregation was greater following treatment with the vitamin C preparation of Example 1 than any of the other formulations.

Leukocyte cell-cell adhesion is associated with xenobiotic induced hyperactivation and inflammatory damage, and vitamin C has been shown to prevent cigarette smoke-induced leukocyte aggregation and attachment to vascular endothelium (Lehr et al., 1994; Weber et al., 1996). As shown in Table 2, vitamin C has also been shown to reduce pesticide mediated T-cell hyperactivation. Given that the formulation of the current invention has greater ability to prevent pesticide-induced T-cell aggregation than other vitamin C formulations, suggests that the formulation of the present invention will provide greater protection against other deleterious xenobiotics.

TABLE 2
The vitamin C preparation of Example 1 inhibits xenobiotic
induced homotypic aggregation in human T-lymphocytes more
effectively than calcium ascorbate-calcium
threonate-dehydroascorbate Ester-C ®.
Activators of T-cell Aggregation

| Vit. C Added | None | PHA | Bifenthrin |
|---|---|---|---|
| None | 10 ± 5 | 170 ± 15 | 300 ± 13 |
| AA | 9 ± 4 | 75 ± 12 | 130 ± 5 |
| CaA | 12 ± 4 | 110 ± 10 | 137 ± 8 |
| EsterC | 8 ± 2 | 120 ± 17 | 200 ± 8 |
| *PWC | 11 ± 6 | 20 ± 9 | 50 ± 10 |

### EXAMPLE 4

The antioxidant and free radical scavenging activity was determined for the vitamin C preparation of Example 1 and known dietary antioxidants.

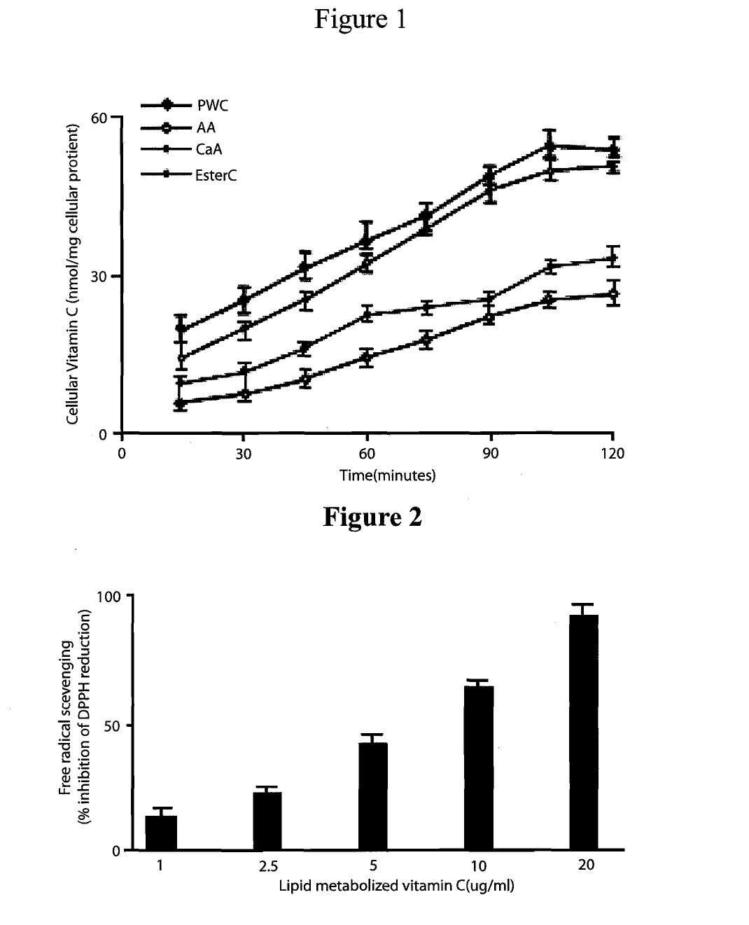

Briefly, 200 ml of a 1, 2.5, 5, 10, or 20 μg/ml solution of the vitamin C preparation of Example 1 was mixed with 50 μl of a 659 μM 1,1-diphenyl-2-picryl hydrazyl (DPPH) solution and incubated at 25° C. for 20 minutes. Free radical scavenging activity of the vitamin C preparation of Example 1 was measured by the reduction of 1,1-diphenyl-2-picryl hydrazyl (DPPH) to 1,1-diphenyl-2-picryl hydrazine at an absorbance of 510 nm. The results are shown in FIG. 2.

The vitamin C preparation dose dependently scavenged DPPH free radicals. The vitamin C preparation demonstrated excellent scavenging ability by reducing the DPPH-induced free radical concentration by 93% at its maximum concentration.

The peroxyl radical oxygen reactive species (ORAC) scavenging ability of the vitamin C preparation was also determined. The ORAC assay detects free radical damage to fluorescein induced by 2,2″-Asobix dihydrochloride (AAPH; 153 mM), and the change is measured by fluorescence spectrophotometry. Antioxidants inhibit the free radical range damage to the fluorescent compound and prevent the reduction in fluorescence. The results are shown in Table 3. The results from different concentrations of the vitamin C preparation of Example 1 were compared to the known antioxidant Trolox®. The ORAC results are expressed as Trolox® equivalents (6-Hydroxy-2,5,7,8-tetramethylchroman-2-carboxylic Acid; TE) per gram of sample.

Vitamin C is a chemical reducing agent for many intracellular and extracellular reactions such as oxidative DNA or protein damage, low-density lipoprotein oxidation, lipid peroxidation, oxidants, the formation of nitrosamines in gastric juice, extracellular oxidants from neutophils, and endothelium dependent vasodilation. The vitamin C preparation of the present invention, which exhibits potent antioxidant and free radical scavenging effects in vitro, can serve as a good vitamin C preparation to prevent such damage thus contributing to the protection against cancer, cardiovascular diseases, atherosclerosis, and other age-related diseases caused by cytotoxic, genotoxic, and proinflammatory mechanisms.

TABLE 3
ORAC values comparing the antioxidant activity of the vitamin C
preparation of Example 1 with known dietary antioxidants.

| Nutrient | ORAC | |
|---|---|---|
| source | (μM TE/g) | Reference |
| The vitamin C preparation | 1343 | Example 4 |
| of Example 1 | | |
| | trial #1 1062 | |
| | trial #2 1394 | |
| | trial #3 1402 | |
| | trial #4 1440 | |
| Cinnamon | 1243 | Sua et al., 2007 |
| Freeze-Dried | 1027 | Schauss et al., 2006 |
| Acai | | |
| Green and | 761.1 | Prior and Cao, 1999 |
| black teas | (235-1526) | |
| Chokeberry | 161 | Wu et al., 2004 |
| Broccoli | 65.8 to 121.6 | Kurilich et al., 2002 |
| Soft wheat | 32-48 | Moore et al., 2005 |
| Careless | 21 | Wu et al., 2004 |
| gooseberry | | |

### EXAMPLE 5

The ability to promote neurite outgrowth was determined for the formulation of Example 1 and other vitamin C formulations.

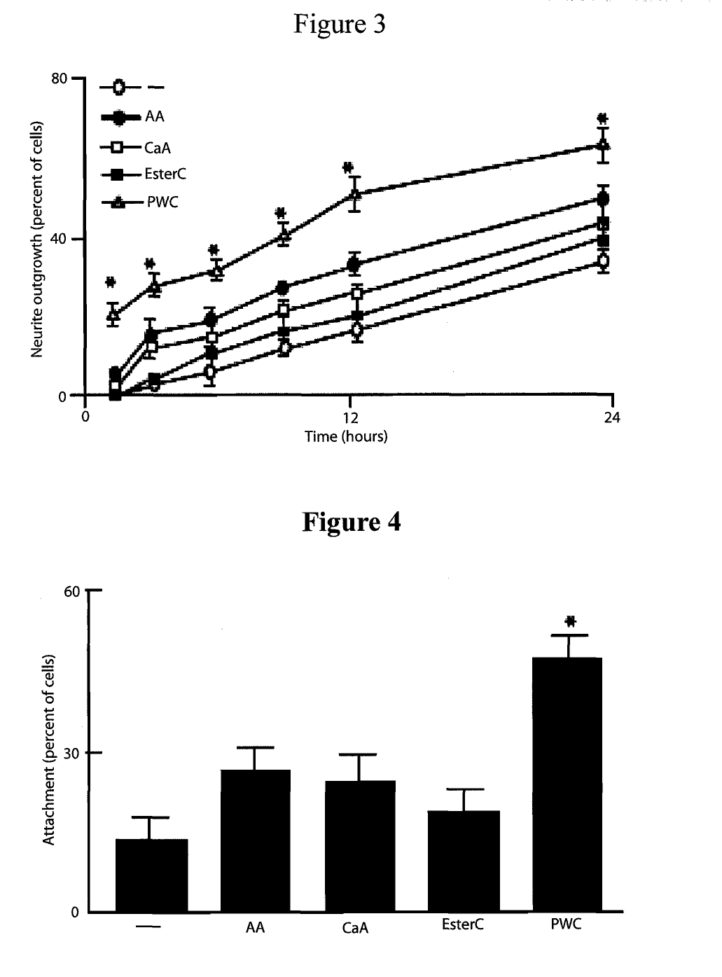

PC12 cells were treated with 100 ng/ml of Nerve Growth Factor (NGF) and incubated for a 24 hour period followed by treatment with either vehicle (-) or various 50 μM of (1) ascorbic acid (AA), (2) calcium ascorbate (CaA), (3) calcium ascorbate-calcium threonate-dehydroascorbate (Ester-C®), or (4) the vitamin C preparation of Example 1 (PWC). The formation of neurites were measured at hours 1, 3, 6, 9, 12, and 24. The results are shown in FIG. 3.

PC12 cells responded to NGF treatment by extending neurites. The vitamin C preparation of Example 1 significantly enhanced the NGF-induced neurite outgrowth in 12% of the cells by the first hour. In fact, the vitamin C preparation was the only formulation that resulted in a significant augmentation of NGF-induced neurite outgrowth, suggesting that this is the only formulation that would aid in protection against neurodegenerative diseases.

### EXAMPLE 6

The ability to promote fibroblast adhesion to fibronectin was determined for the formulation of Example 1 and other vitamin C formulations.

NIH3T3 fibroblastoma cells were seeded onto fibronectin coated plates pretreated with either vehicle (-) or various 50 mM of (1) ascorbic acid (AA), (2) calcium ascorbate (CaA), (3) calcium ascorbate-calcium threonate-dehydroascorbate (Ester-C®), or (4) the vitamin C preparation of Example 1 (PWC). The plates were incubated for 15 minutes at 37° C. The unattached cells were removed by aspiration and the attached cells were fixed, stained, and counted in triplicate. Results are shown in FIG. 4.

The vitamin C preparation of Example 1 enhanced fibroblast adhesion to fibronectin by over three-fold. In addition to adhesion, fibroblast spreading on fibronectin is an important next step to migration and wound healing performance.

### EXAMPLE 7

The human serum vitamin C, plasma C-reactive protein, oxidized LDL, and urine uric and oxalate levels were determined for the formulation of Example 1 and other vitamin C formulations.

Healthy volunteers maintained a low vitamin C diet for 14 days. Following an overnight fast, volunteers received a single oral dose of 1000 mg or either (1) ascorbic acid (AA), (2) calcium ascorbate (CaA), (3) calcium ascorbate-calcium threonate-dehydroascorbate (commercially available as Ester-C® from Nature's Value of Coram, N.Y.) (Ester-C®), or (4) the vitamin C preparation of Example 1 (PWC). Blood samples were collected immediately prior to the oral dose administration and at various time points post ingestion. Urine was collected over a 24-hour time period and saved for oxalate and uric acid assays. Serum vitamin C levels were measured by HPLC with coulometric electrochemical detection. Plasma C-reactive protein and oxidized LDL were measured by enzyme linked immunosorbent assay (ELISA) and urine uric acid and oxalate levels were measured by enzymatic methods.

The vitamin C preparation of Example 1 is more rapidly absorbed and leads to higher serum vitamin C levels and greater reduction of plasma levels of inflammatory and oxidative stress markers than other forms of vitamin C.

TABLE 4
Clinical data comparing the serum vitamin C levels, plasma C-reactive protein,
oxidized LDL levels, and urine uric acid and oxalate levels of the vitamin C preparation
of Example 1 with other vitamin C formulations.
Serum Vitamin C Levels
(mg/dl)

| | Hrs Post-Admin: | | | | | |
|---|---|---|---|---|---|---|
| Vitamin C | 0 | 1 | 2 | 4 | 6 | 24 |
| AA | 0.56 ± 0.06 | 1.2 ± 0.10 | 1.64 ± 0.18 | 1.51 ± 0.22 | 1.46 ± 0.13 | 0.80 ± 0.09 |
| CaA | 0.50 ± 0.05 | 0.88 ± 0.10 | 1.12 ± 0.17 | 1.03 ± 0.13 | 1.0 ± 0.13 | 0.59 ± 0.09 |
| EsterC | 0.56 ± 0.09 | 1.3 ± 0.08* | 2.17 ± 0.19* | 1.54 ± 0.14* | 1.51 ± 0.19* | 0.85 ± 0.08 |
| *PWC | 0.60 ± 0.08 | 1.22 ± 0.11* | 1.69 ± 0.27 | 1.52 ± 0.16* | 1.17 ± 0.12 | 0.73 ± 0.07 |

| | Plasma C-Reactive Protein | | | Plasma OxLDL | | | Urine Markers | |
|---|---|---|---|---|---|---|---|---|
| | (ng/ml) | | | (U/ml) | | | (mg/dl) | |
| | 0 | 24 | Change | 0 | 24 | Change | Uric Acid | Oxalate |
| AA | 129.75 ± 26 | 117.00 ± 33 | 12.75 | 68.78 ± 6 | 67.89 ± 5 | 0.89 | 50.85 ± 8.8 | 18.8 ± 2.7 |
| CaA | 189.17 ± 41 | 180.83 ± 43 | 8.34 | 60.56 ± 5 | 57.67 ± 6 | 3.78 | 39.75 ± 10.5 | 17.8 ± 2.6 |
| EsterC | 152.30 ± 19 | 128.60 ± 19 | 23.7 | 62.56 ± 5 | 57.30 ± 4 | 5.26** | 48.73 ± 7.1 | 13.7 ± 1.5 |
| *PWC | 200.63 ± 38 | 180.00 ± 52 | 20.63 | 50.51 ± 4 | 48.20 ± 4 | 2.31 | 40.96 ± 7.0 | 17.9 ± 1.9 |

*Statistically significant deference compared to Calcium Ascorbate. At one hour p = 0.0026 for PWC and p = 0.049 for Ester-C. At two hours, p = 0.0009. At four hours p = 0.0278 for PWC and 0.0477 for Ester C. At six hours, p = 0.0470
**Statistically significant difference from Ascorbic Acid (p = 0.045). Note that the reductions in oxLDL were not significantly different for any vitamin C supplementation with a before-and-after comparison; however, the drop observed with PWC was significantly greater than the drop observed with Ascorbic Acid.
Note:
All statistically significant differences are noted. Data are presented as the mean + S.E.M. All 0 time points were immediately prior to oral administration of the vitamin C formulation

### REFERENCES

Bessey O, Lowry O, Brock M: The quantitative determination of ascorbic acid in small amount of white blood cells and platelets. JBC 1947, 168(1):197-205
Boothby L A, Doering P L: Vitamin C and vitamin E for Alzheimer's disease. Ann. Pharmacotherapy 2005, 39(12):2073-80
Fay M J, Bush M J, Verlangieri A J: Effect of aldonic acids on the uptake of ascorbic acid by 3T3 mouse fibroblasts and human T lymphoma cells. Gen. Pharmacol. 1994, 25(7):1465-69
Kaplan B, Gonul B, Dincer S, Dincer Kaya F N, Babul A: Relationships between tensile strength, ascorbic acid, hydroxyproline, and zinc levels of rabbit full-thickness incision wound healing. Surg. Today 2004, 34(9):747-51
Kurilich A C, Jefferey E H, Juvik J A, Wallig M A, and Klein B P: Antioxidant capacity of different broccoli (Brassica oleracea) genotypes using the oxygen radical absorbance capacity (ORAC) assay. J. Agric. Food Chem. 2002, 50(18):5053-57
Landmark K: Could intake of vitamins C and E inhibit development of Alzheimer dementia? Tidsskr Nor Laegeforen 2006, 15(8):159-61
Lehr H A, Frei B, Arfors K E: Vitamin C prevents cigarette smoke-induced leukocyte aggregation and adhesion to endothelium in vivo. PNAS 1994, 91(16):7688-92
Marionnet C, Vioux-Chagnoleau C, Pierrard C, Sok J, Asselineau D, Bernerd F: Morphogenesis of dermal-epidermal junction in a model of reconstructed skin: beneficial effects of vitamin C. Exp. Dermatol. 2006, 15(8):625-33
Moore J, Hao Z, Zhou K Luther M, Costa J, Y L: Carotenoid, tocopherol, phenolic acid, and antioxidant properties of Maryland-grown soft wheat. J. Agric. Food Chem. 2005, 53(17):6649-57
Prior R L, Cao G: Antioxidant capacity and polyphenolic components of teas: implications for altering in vivo antioxidant status. Proc. Soc. Exp. Biol. Med. 1999, 220(4):255-61
Schauss A G, Xianli W., Prior R L, Ou B, Huang D, Owens J, Agarwal A, Jensen G S, Hart A N, Shanbrom E: Antioxidant capacity and other bioactivities of the freeze-dried Amazonian palm berry, Euterpe oleraceae Mart. (Acai). J. Agric Food Chem. 2006, 54(22):8604-10
Su L, Yin J-J, Charles D, Zhou K, Moore J, and Yu L: Total phenolic contents, chelating capacities and radical-scavenging properties of black peppercorn, nutmeg, rosehip, cinnamon and oregano leaf. Food Chem. 2007, 100(3):990-97
Weber C, Erl W, Weber K, Weber P C: Increased adhesiveness of isolcated monocytes to endothelium is prevented by vitamin C intake in smokers. Circulation 1996, 93(8):1488-92
Weeks B S and Perez P P: A novel vitamin C preparation enhances neurite formation and fibroblast adhesion and reduces xenotiotic-induced T-cell hyperactivation. Med. Sci. Monit. 2007, 13(3):BR51-58
Wu X, Gu L, Prior R L, McKay S: Characterization of anthocyanins and proanthocynaidins in some cultivars of Ribes, Aronia, and Sambucus and their antioxidant capacity. J. Agric. Food Chem. 2002, 52(26):7846-56
Zhou X, Tai A, Yamamotol: Enhancement of neurite outgrowth in PC12 cell stimulated with cyclic AMP and NGF by 6-acylated ascorbic acid 2-O-alpha-glucosides (6-Acyl-AA-2G), novel lipophilic ascorbate derivatives. Biol. Pharm. Bull. 2003, 26(3):341-46.

We claim:

1. A vitamin C preparation comprising:
(a) at least about 90% by weight of vitamin C,
(b) at least about 0.1% by weight of lipophilic molecules comprising (i) one or more saturated straight C₃₀-C₃₄ fatty alcohols, (ii) one or more unsaturated ω-9 C₁₈-C₂₄ fatty acids, (iii) optionally one or more saturated straight C₁₄-C₂₀ fatty acids, (iv) optionally one or more unsaturated ω-3 C₁₆-C₂₄ fatty acids, and (v) optionally one or more unsaturated ω-6 C₁₈-C₂₂ fatty acids; and
(c) optionally at least about 0.1% by weight of bioflavonoids, based upon 100% total weight of the vitamin C preparation.

2. The vitamin C preparation of claim 1, wherein the vitamin C preparation comprises (i) at least about 0.1% by weight of one or more saturated straight C₃₀-C₃₄ fatty alcohols, (ii) at least about 0.01% by weight of component one or more unsaturated ω-9 C₁₈-C₂₄ fatty acids, (iii) optionally at least 0.01% by weight of one or more saturated straight C₁₄-C₂₀ fatty acids, (iv) optionally at least about 0.01% by weight of one or more unsaturated ω-3 C₁₆-C₂₄ fatty acids, and (v) at least about 0.01% by weight of one or more unsaturated ω-6 C₁₈-C₂₂ fatty acids, based upon 100% total weight of vitamin C preparation.

3. The vitamin C preparation of claim 1, wherein the vitamin C preparation comprises (a) at least 90% by weight of vitamin C, (b) 0.1 to 5% by weight of the lipophilic molecules, and (c) 0.1 to 5% by weight of the bioflavonoids.

4. The vitamin C preparation of claim 1, wherein the vitamin C preparation comprises about 200 to 40,000 IU vitamin C.

5. The vitamin C preparation of claim 1, wherein the vitamin C preparation comprises the following lipophilic molecules:
about 0.01-0.3% (by weight) palmitic acid,
about 0.01-2.0% linoleic acid,
about 0.01-0.6% alpha linolenic acid,
about 0.01-0.4% oleic acid,
about 0.1-0.8% steric acid,
about 0.01-0.09% arachidic acid,
about 0.01-0.09% heneicosanoic acid,
about 0.1-0.9% behenic acid,
about 0.1-0.9% tricosanoic acid,
about 0.01-0.9% lignoceric acid,
about 0.05-0.9% cerotic acid,
about 0.1-1.0% heptacosanoic acid,
about 0.05-1.5% montanic acid,
about 0.2-2.6% melissic acid,
about 0.05-1.6% docosahexaenoic acid,
about 0.05-0.9% docosapentaenoic acid,
about 0.05-1.9% docosatetraenoic acid,
about 0.05-0.9% docosadienoic acid,
about 0.01-1.8% erucic acid,
about 0.01-0.09% nervonic acid,
about 0.01-8.0% cetyl alcohol-hexadecanol-palmityl alcohol,
about 0.01-5.0% 1-heptadecanol,
about 0.01-1.0% 1-eicosanol-arachidyl alcohol,
about 0.01-3.0% 1-docosanol-behenyl alcohol,
about 1.0-15.0% lignoceryl alcohol-1-tetracosanol,
about 1.0-12.0% 1-hexacosanol-ceryl alcohol,
about 0.01-2.0% 1-heptacosanol,
about 0.5-20.0% 1-octacosanol,
about 15.0-40.0% 1-triacontanol-melissyl alcohol,
about 10.0-20.0% dotriacontanol, and
about 5.0-15.0% tetratriacontanol
based upon 100% total weight of the lipophilic molecules in the vitamin C preparation.

6. The vitamin C preparation of claim 1, wherein the lipophilic molecules are extracted from a natural wax.

7. The vitamin C preparation of claim 5, wherein the wax is selected from sugar cane wax, rice bran wax, carnauba wax, candelilla wax, japan wax, ouricury wax, bayberry wax, shellac wax, sunflower wax, orange wax, and beeswax.

8. The vitamin C preparation formulation of claim 1, wherein the bioflavonoids are a mixture comprising hesperidin and gallic acid.

9. The vitamin C preparation of claim 7, wherein the vitamin C preparation comprises the following bioflavonoids:
about 20-120 ppm rutin,
about 25-100 ppm naringin,
about 7000-20000 ppm hesperidin,
about 5-100 ppm neohesperidin,
about 10-100 ppm neohesperidin dihydrochalcone,
about 5-100 ppm naringenin,
about 5-100 ppm hersperitin,
about 50-150 ppm nomilin, and
about 120-1000 mg/g gallic acid,
based upon 1 g of the bioflavonoids.

10. The vitamin C preparation of claim 1, wherein the vitamin C is ascorbic acid or a pharmaceutically acceptable salt thereof.

11. A method of promoting a healthy nervous system in a human comprising:
(a) recognizing the vitamin C preparation of claim 1 as being effective to promote a healthy nervous system, and
(b) after such recognition, orally administering to the human an effective amount of the vitamin C preparation of claim 1.

12. A method of decreasing the risk of a human of developing a neurodegenerative disease comprising:
(a) recognizing the vitamin C preparation of claim 1 as being effective to decrease the risk of a human of developing a neurodegenerative disease, and
(b) after such recognition, orally administering to the human an effective amount of the vitamin C preparation of claim 1.

13. A method of enhancing NGF-mediated neurite outgrowth in a human comprising:
(a) recognizing the vitamin C preparation of claim 1 as being effective to enhance NGF-mediated neurite outgrowth, and
(b) after such recognition, orally administering to the human an effective amount of the vitamin C preparation of claim 1.

14. A method of promoting wound healing in a human comprising:
(a) recognizing the vitamin C preparation of claim 1 as being effective to promote wound healing, and
(b) after such recognition, orally administering to the human an effective amount of the vitamin C preparation of claim 1.

15. A method of enhancing fibroblast adhesion to and the interaction with the extracellular matrix in a human comprising:
(a) recognizing the vitamin C preparation of claim 1 as being effective to enhance fibroblast adhesion to and the interaction with the extracellular matrix, and
(b) after such recognition, orally administering to the human an effective amount of the vitamin C preparation of claim 1.

16. A method of protecting the immune system from xenobiotics in a human comprising:
(a) recognizing the vitamin C preparation of claim 1 as being effective to protect the immune system from xenobiotics, and
(b) after such recognition, orally administering to the human an effective amount of the vitamin C preparation of claim 1.

17. A method of decreasing the risk of developing an oxidative pathogenesis in a human comprising:
(a) recognizing the vitamin C preparation of claim 1 as being effective to decrease the risk of a human of developing an oxidative pathogenesis, and
(b) after such recognition, orally administering to the human an effective amount of the vitamin C preparation of claim 1.

18. A method of decreasing the risk of developing cancer, cardiovascular diseases, atherosclerosis, and other age-related diseases associated with cytotoxic, genotoxic, and proinflammatory mechanisms in a human comprising:
(a) recognizing the vitamin C preparation of claim 1 as being effective to decrease the risk of a human of cancer, cardiovascular diseases, atherosclerosis, and other age-related diseases associated with cytotoxic, genotoxic, and proinflammatory mechanisms, and
(b) after such recognition, orally administering to the human an effective amount of the vitamin C preparation of claim 1.
